(19) United States
(12) Patent Application Publication
(10) Pub. No.: US 2007/0031480 A1
(43) Pub. Date: Feb. 8, 2007

(54) Enhanced delivery of sphingolipids

(76) Inventors: Lawrence Mayer, North Vancouver (CA); Jennifer Shabbits, Richmond (CA); Marcel Bally, Bowen Island (CA)

Correspondence Address: MORRISON & FOERSTER LLP, 12531 HIGH BLUFF DRIVE, SUITE 100, SAN DIEGO, CA, 92130-2040, US

(21) Appl. No.: 10/548,371

(22) PCT Filed: Mar. 8, 2004
(86) PCT No.: PCT/CA04/00344

§ 371 Date: Sep. 29, 2006

Related U.S. Application Data
(60) Provisional application No. 60/453,002, filed on Mar. 7, 2003.

(30) Foreign Application Priority Data
Mar. 7, 2003 (CA) 2,421,316

(51) Int. Cl. A61K 9/127; A61K 31/685; A61K 31/56; A61K 31/739

(52) U.S. Cl. 424/450; 514/78; 514/171; 977/907; 514/54

(57) ABSTRACT

Compositions which comprise delivery vehicles having stably associated therewith at least one organic acid derivative of a sterol are useful in achieving enhanced cellular delivery of physiologically relevant sphingolipids when the compositions are administered.

### CROSS-REFERENCE TO RELATED APPLICATIONS

This application claims benefit of U.S. application Ser. No. 60/453,002 filed 7 Mar. 2003. The contents of this application are incorporated herein by reference.

### TECHNICAL FIELD

The invention relates to compositions and methods for improved delivery of sphingolipids, such as ceramide. More particularly, the invention concerns delivery systems that ensure the intracellular uptake of effective amounts of sphingolipids when the sphingolipids are delivered to an intended target by providing a formulation comprising delivery vehicles.

### BACKGROUND ART

The progression and treatment of many life-threatening diseases such as cancer, immune disorders and cardiovascular disorders is influenced by multiple molecular mechanisms, including those that regulate the balance between cell proliferation and cell death. In particular, hyperproliferative disorders such as cancer result from an increase in cell proliferation that is often coupled with a decrease in programmed cell death (apoptosis). Key molecules being examined for their role in regulating intracellular processes that affect this balance are ceramides and related sphingolipids such as sphingosine. These molecules are known to be involved in regulating a number of cellular responses to both environmental and pharmacological extracellular stimuli. Such responses include, differentiation, inhibition of growth, cell senescence and apoptosis (Kolesnick, R. N., and Kronke, M., Annu. Rev. Physiol. (1998) 60:643-665; Hannun, Y. A., Science (1996) 274:1855-1859; and Ariga, T., et al., J. Lipid Res. (1998) 39:1-16).

The role of ceramide in apoptosis and reduced growth may be particularly important for the treatment of cancer. One dramatic demonstration of the relationship between ceramide and cancer was reported by Selzner and colleagues who showed that the cellular content of ceramide in human colon cancer is reduced by more than 50% relative to that of healthy colon mucosa (Selzner, et al., Cancer Res. (2001) 61(3):1233-1240). Similarly, alterations in metabolism of ceramide to the noncytotoxic metabolite glucosylceramide have been implicated in the multidrug resistance (MDR) phenomenon associated with numerous cancers (Shabbits and Mayer, Mol. Cancer Ther. (2002) 1(3):205-213).

Ceramides are a class of lipid second messengers comprising a sphingosine backbone and are found in all eukaryotic membranes. They are generated within a cell from the hydrolysis of sphingomyelin or de novo biosynthesis. Different ceramides are characterized by different fatty acids linked to the sphingoid base. Pei, et al., WO 95/21175, have shown that analogs of sphingolipids and ceramides that inhibit conversion of ceramides to sphingomyelins lead to enhanced apoptosis as a result of the increased intracellular ceramide content. In fact, much of the research focusing on ceramides has been directed at altering their intracellular levels. Of particular interest is the observation in several systems that transformed cell types are hypersensitive to the effects of ceramide perturbation, suggesting that strategies to kill tumor cells by increasing their ceramide content should have a favorable therapeutic index.

Increasing intracellular ceramide levels has been achieved with multiple endogenous methods that inhibit ceramide catabolism, such as those performed by Pei, et al., as well as exogenous administration of cell-permeable (short-chain) ceramides. Traditionally, these short-chain ceramides (C₂ or C₆) are efficiently incorporated into liposomes comprised of phosphatidylcholine or phosphatidylethanolamine and are effectively delivered to the cytosol of a target cell. Pei, et al., showed that C₂- and C₆-ceramide or related analogs, incorporated into various liposomal formulations comprising egg phosphatidylcholine or sphingqmyelin and cholesterol, exhibited significant cytotoxic effects both in vitro and in vivo.

More recently, however, it has been demonstrated in vitro that the effects of short-chain ceramides on the activity of particular intracellular molecules are different from those of natural ceramide, indicating that the cell-permeable short-chain ceramide compounds many not completely mimic the natural product (Huang, et al., Biophys. J. (1999) 77:1489-1497). Thomas, et al., have also indicated that the ceramide species involved in apoptosis of Jurkat cells is the long-chain, C₁₆-ceramide (Thomas, et al., J Biol. Chem. (1999) 274:30580-30588).

Research relating to the naturally occurring ceramides and other long-chain sphingolipids has been limited because of their hydrophobic nature making them cell-impermeable and also difficult to formulate at effective amounts into delivery vehicles, such as liposomes. Physicochemical characterization of long-chain ceramides in lipid bilayers have shown that greater than about 20 mol % of these ceramides leads to extensive liposomal aggregation. Holopainen, et al., Chem. Phys. of Lipids (1997) 88:1-13) prepared dimyristoylphosphatidylcholine (DMPC) or 1-palmitoyl-2[(pyren-1-yl)]decanoyl-sn-glycero-3-phosphocholine (PPDPC) liposomes incorporating ceramides ranging from C₁₆-C₂₇ at up to 20 mol % in order to study thermal phase behaviors with Differential Scanning Calorimetry and Fluorescence Spectroscopy. Liposomes incorporating more than 20 mol % could not be studied due to extensive aggregation. The researchers did not investigate alternate liposome formulations that could relieve this aggregation nor did they determine if the ceramide-containing liposomes exhibited a cytotoxic effect. Similarly, using Nuclear Magnetic Resonance imaging, Hsueh and colleagues examined the effect of increasing ceramide concentrations on 1-palmitoyl-2-oleoyl-sn-glycero-3-phosphocholine (POPC) membranes (Hsueh, et al., Biophys. J. (2002) 82:3089-3095). They found that increasing the level of C₁₆-ceramide above 20 mol % resulted in the formation of unstable (or metastable) liposomes which exhibited significant hydration complexities.

Despite the many liposomal formulations designed for delivery of sphingolipids, a composition suitable for long-chain sphingolipids has not been achieved. It is especially important to accomplish this since it has been suggested that manipulation of ceramide levels may also enhance the effectiveness of some cancer therapies.. Wanebo and coworkers showed that the addition of ceramide enhances taxol-mediated apoptotic death of Tul 38 head and neck tumor cells (Mehta, et al., Cancer Chemother. Pharmacol. (2000) 46(2):85-92). Because a variety of anti-cancer drugs are known to elevate endogenous ceramide levels, combinations of sphingolipids with anti-cancer drugs may result in significantly improved chemotherapy regimes.

### DISCLOSURE OF THE INVENTION

It has been found that formulations of the present invention can incorporate as much as 50 mole % C₁₆-ceramide without aggregation and that these formulations result in a significant Increase in Life Span (ILS) of a host when administered in vivo. The delivery vehicles described herein provide effective delivery of sphingolipids, even those that are substantially insoluble in water. The delivery vehicles comprise at least one acid derivative of a sterol.

Thus, in one aspect, the invention relates to methods for administering sphingolipids, including hydrophobic sphingolipids, using delivery vehicle compositions comprising an acid derivative of a sterol. Incorporation of the acid-derivatized sterol in the delivery vehicle allows for increased encapsulation of sphingolipids without causing aggregation of the delivery vehicles, resulting in enhanced intracellular delivery.

In another aspect, the invention provides a delivery vehicle-containing composition for administration comprising an effective amount of a sphingolipid and an acid derivative of a sterol. Sphingolipids may include long-chain, hydrophobic ceramides. Another aspect of the invention is directed to a method to deliver an effective amount of a sphingolipid to a desired target by administering the compositions of the invention.

The present invention provides liposomes incorporating at least one acidic lipid and a sphingolipid. The inclusion of an acidic lipid allows the liposome to remain stable at physiological pH and to destabilize upon delivery to a low pH target site such as endosomes and tumors. Destabilization of the liposome allows for increased availability of the sphingolipid at a target site. In preferred embodiments, delivery of the sphingolipid is enhanced by the incorporation of a lipid that has a net negative charge at physiological pH and neutral at reduced pH. Conversion of the acidic lipid to its neutral form at low pH triggers liposome destabilization thereby allowing for release of the sphingolipid from the bilayer along with encapsulated contents, if present. Preferably the acidic lipid is a derivatized sterol. More preferably, the acid lipid is cholesteryl hemisuccinate.

The delivery vehicles may further comprise one or more encapsulated active agents.

### BRIEF DESCRIPTION OF THE DRAWINGS

FIG. 1 is a graph showing the cytotoxicity of various acyl chain length free ceramide lipids on wild-type (A) and MDR-1 gene transfected (B) MDA435/LCC6 human breast cancer cells. Cells were incubated with the indicated ceramide concentrations for 72 hours and cell viability was measured using the MTT assay. Data are averaged means from three triplicate experiments. Each value represents the mean from at least three independent experiments; error bars indicate the Standard Error of the Mean (S.E.M).

FIG. 2 is a graph showing the cytotoxicity of various acyl chain length free ceramide lipids on J774 murine macrophage cells. Cells were incubated with the indicated ceramide concentrations for 72 hours and cell viability was measured using the MTT assay. Data are averaged means from three triplicate experiments. Each value represents the mean from at least three independent experiments; error bars indicate the S.E.M.

FIG. 3 is a graph showing the cellular uptake of free C₆- and C₁₆-ceramide by wild-type (A) and MDR-1 gene transfected (B) MDA435/LCC6 cells. Cells were incubated with 10 μM C₆-ceramide or 50 μM C₁₆-ceramide for the times indicated. [¹⁴C]C₆- or [¹⁴C]C₁₆-ceramide was added at 0.1 μCi/nmole ceramide for quantitation by scintillation counting. Cellular protein content was measured spectrophotometrically (Abs 562 nm) using the micro BCA protein assay kit. Data are averaged means from two triplicate experiments; error bars indicate the S.E.M.

FIG. 4 is a graph showing the cytotoxicity of control (DPPC/CHEMS, 50:50) and C₁₆-ceramide (C₁₆-ceramide/CHEMS, 50:50) liposomes on J774 murine macrophage cells. Cells were incubated with the indicated concentrations of liposomes for 72 hours and cell viability was measured using the MTT assay. The indicated liposome concentration represents total lipid for control liposomes and was corrected for ceramide content for ceramide-containing liposomes. Data are averaged means from three triplicate experiments; error bars indicate the S.E.M.

FIG. 5 is a graph showing the cellular uptake of C₁₆-ceramide/CHEMS (50:50) liposomes by J774 murine macrophage cells. Uptake of [³H]cholesterylhexadecyl ether ([³H]CHE) bulk liposomal lipid and [¹⁴C]C₁₆-ceramide are expressed as a percent of the total radioactivity added, normalized to 10⁵ cells. Data are averaged means from two triplicate experiments; error bars indicate the S.E.M.

FIG. 6 is a graph showing the antitumor activity of C₁₆-cer/CHEMS/PEG2000-DSPE (47.5:47.5:5) versus DPPC/CHEMS/PEG2000-DSPE (47.4:47.5:5) liposomes in the J774 ascites tumor model. On day zero, 1×10⁶ cells were inoculated intraperitoneally (i.p.) into female Balb/c mice (12 per group) and saline, control liposomes or ceramide liposomes were administered by intravenous (i.v.) bolus injection on days 1, 5 and 9 at the lipid concentrations indicated. Arrows indicate the days of treatment administration. Animals were weighed and monitored daily for survival.

FIG. 7 is a graph showing the antitumor activity of C₁₆-cer/CHEMS/PEG2000-DSPE (47.5:47.5:5) versus DPPC/CHEMS/PEG2000-DSPE (47.4:47.5:5) liposomes in the J774 ascites tumor model. On day zero, 1×10⁶ cells were inoculated intraperitoneally (i.p.) into female Balb/c mice (12 per group) and saline, control liposomes or ceramide liposomes were administered i.p. on days 1, 5 and 9 at the lipid concentrations indicated. Arrows indicate the days of treatment administration. Animals were weighed and monitored daily for survival.

### MODES OF CARRYING OUT THE INVENTION

The invention provides compositions comprising delivery vehicles that include at least one acid-derivative of a sterol that are useful in delivering sphingolipids, especially long-chain sphingolipids. The acid-derivative sterol aids in incorporating high levels of said sphingolipid. The acid-derivatized sterol may be pH sensitive in that it is negative at physiological pH and neutral at lower pH.

Preferably delivery vehicles included herein will incorporate high levels of sphingolipids. The delivery vehicles will contain greater than 20 mol % of sphingolipids, more preferably, greater than 30 mol % of said sphingolipids, more preferably more than 40 mol % or 50 mol %, the base for these percentages is total lipid.

In further embodiments of the invention, the above described delivery vehicles incorporate one or more additional active agents. Any therapeutic, cosmetic or diagnostic agent may be included.

In another aspect of the invention, delivery vehicles which comprise a pH sensitive, “acidic lipid”, and a sphingolipid are provided.

The delivery vehicles of the present invention may be used not only in parenteral administration but also in topical, nasal, subcutaneous, intraperitoneal, intramuscular, or oral delivery or by the application of the delivery vehicle onto or into a natural or synthetic implantable device at, or near the target site for therapeutic purposes or medical imaging and the like. Preferably, the delivery vehicles of the present invention are used in parenteral administration, most preferably, intravenous administration.

The preferred embodiments herein described are not intended to be exhaustive or to limit the scope of the invention to the precise forms disclosed. They are chosen and described to best explain the principles of the invention and its application and practical use to allow others skilled in the art to comprehend its teachings.

Abbreviations

DMPC: dimyristoylphosphatidylcholine;
PPDPC: 1-palmitoyl-2[pyren-1-yl)]decanoyl-sn-glycero-3-phosphocholine;
POPC: 1-palmitoyl-2-oleoyl-sn-glycero-3-phosphocholine;
DSPC: distearoylphosphatidylcholine; DSPE: distearoylphosphatidylethanolamine;
DOPE: dioleoylphosphatidylethanolamine; DPPC: dipalmitoylphosphatidylcholine;
DPPG: 1,2-dipalmitoyl-sn-glycero-3-[phospho-rac-(1 -glycerol)]; DSPE-PEG350 or
PEG350-DSPE: distearoylphosphatidylethanolamine-N-[polyethylene glycol 350].

Chol or CH: cholesterol; CHEMS or CHS: cholesteryl hemisuccinate; cer: ceramide.

Acid-Derivatized Sterols

“Acid-Derivatized Sterol” refers to a steroid that is coupled to an acidic group—i.e., a group that is negatively charged at physiological pH. Preferably the steroid contains a hydroxyl group and the acid to be coupled is sufficiently bifunctional to form an ester with the alcohol and retain its acidic characteristics. However, other coupling means may be used.

Cholesterol and other sterols are routinely used in delivery vehicle preparations, such as liposomes, in order to broaden the range of temperatures at which phase transition occurs, with phase transition disappearing at high cholesterol levels. Many acid-derivatized sterols have also been incorporated into a number of cosmetic, diagnostic and pharmaceutical preparations. Particular acid-derivatized cholesterols, such as cholesteryl phosphate and cholesteryl hemisuccinate, are known to retain many of the properties that cholesterol exhibits in model membrane systems (Lai, et al., Biochemistry (1995) 24(7):1646-1653). However, these cholesterol esters also display unique properties in the extent to which they interact or bind with phospholipids (Lai, et al., Biochemistry (1995) 24(7):1654-1661). The attachment of the charged ester is known to greatly enhance the partitioning of cholesterol and other sterols into phospholipid bilayers. This has been beneficial for many cosmetic formulations that have incorporated hydrophilic sterols (see European Patent No. 28,456 and U.S. Pat. No. 4,393,044).

It is well known that many acid-derivatized sterols can readily self-assemble into multi- or single lamellar vesicles without the use of organic solvents. These vesicles display high trapping efficiencies and captured volumes. The present invention describes delivery vehicle compositions comprised of at least one acid-derivatized sterol, preferably an organic acid derivative of cholesterol, which allows for enhanced incorporation of sphingolipids into said delivery vehicles. Preferably, the derivatized sterol is capable of self-assembling into a closed bilayer; however, any acid-derivatized sterol may be used in the practice of the invention. The suitability of a particular sterol derivative depends upon its ability to allow for stable association of high levels of sphingolipids, preferably hydrophobic sphingolipids, with delivery vehicles of the invention. Generally any sterol which can be modified by the attachment of an organic acid may be used in the practice of the invention. Non-limiting examples of such sterols include cholesterol, Vitamin D, phytosterols, steroid hormones and the like.

Organic acids which can be used to derivatize the sterols include, but are not limited to, di- and polycarboxylic acids, hydroxy acids, amino acids and polyamino acids. Particular organic acid moieties that are water-soluble themselves may be more advantageous in increasing the hydrophilicity of the acid-derivatized sterol. Non-limiting examples of such moieties include dicarboxylic acids such as malonic, succinic, glutaric, adipic, pimelic, maleic and the like; and aromatic dicarboxylic acids such as hemimellitic, trimesic, and the like; hydroxy acids such as glycolic, lactic, mandelic, glyceric, malic, tartaric, citric and the like; and any amino or polyamino acid. The derivatized acid can be linked to the hydroxyl group of the sterol preferably via an ester bond using conventional methods (see for example, U.S. Pat. Nos. 3,859,047; 4,040,784 or 4,189,400). If the carboxylic acid contains only a single carboxyl group, other reactive groups present in the molecule can be used to couple the acid moiety to any reactive functional group on the sterol. In some cases, such as formation as phosphate esters, the multivalent nature of the acid itself is sufficient.

A further advantage of acid-derivatized sterols is their sensitivity to pH. Generally, such sterol derivatives have a net negative charge at physiological pH which enhances the stability of delivery vehicles incorporating them. Therefore, delivery vehicles of the invention remain stable in the blood compartment (physiological pH) and destabilize upon delivery to a low pH target site, such as endosomes or tumors, where they become protonated. This destabilization allows for release of the contents, which may allow more rapid uptake. It should be apparent to those knowledgeable in the art that other acidic lipids, with similar pH sensitivities, could be included in the vehicles of this invention. Thus another aspect of the invention provides delivery vehicles comprising an acidic lipid and a sphingolipid. Preferably the acidic lipid is a sterol derivative. More preferably, the acidic lipid is a derivative of cholesterol. Even more preferably, the acidic lipid is cholesteryl hemisuccinate.

Sphingolipids

Sphingolipids, as described herein, are compounds that comprise long-chain bases containing a secondary amine and one or more hydroxyl groups. The most commonly found long-chain bases are sphingosine (4-sphingenine), sphinganine and 4-hydroxy sphinganine. Sphingosine is most commonly found in mammalian cells and has the formula CH₃(CH₂)₁₂CH═CHCHOHCHNH⁺₃CH₂OH. The other two long-chain bases are those wherein the CH═CH is reduced to —CH₂CH₂—or hydrated to —CH₂CHOH. The latter two are commonly called dihydrosphingosine and phytosphingosine; as implied by the name, the last named long-chain base is most commonly found in plants. These long-chain bases can, themselves, be considered sphingolipids, but most commonly, sphingolipids include forms of these bases wherein the amino and/or hydroxyl groups are derivatized. The ceramides are members of the sphingolipid class which are acylated at the amino group. In addition, one or more hydroxyl groups, typically the primary hydroxyl group, can further be derivatized, for example with phosphocholine to yield sphingomyelin.

As used herein, “sphingolipid” there refers to the derivatized and underivatized forms of sphingosine and its related compounds which have the essential features of containing a secondary amine and at least one hydroxyl group. Included in the sphingolipids, therefore, are derivatives of sphingosine, derivatives of phytosphingosine, derivatives of dihydrosphingosine, and related long-chain bases and specifically includes the ceramides. The ceramides are defined as sphingolipid derivatives which comprise acyl groups coupled to the amino group to form an amide. “Derivatives of ceramide” refer to further substitutions on the hydroxyl groups of ceramides. They are also sphingolipids.

In some instances, the derivatives of sphingosine and ceramide or of other long-chain bases are designed to block the metabolism of ceramides. This function is useful in enhancing the levels of ceramides in cells, and thus enhancing the apoptotic potential of these cells. Accordingly, these derivatives, that block ceramide metabolism, are useful antitumor agents.

Sphingolipids may be hydrophilic or hydrophobic. Hydrophobic sphingolipids generally contain greater than 6 carbon atoms in at least one acyl chain. Sphingolipids that induce apoptosis or sphingolipids that mediate opposing pathways may be used. Preferably sphingolipids of the invention are hydrophobic. More preferably they are long-chain ceramides or ceramide derivatives. Even more preferably, they are therapeutically active and physiologically relevant ceramides or derivatives thereof.

Many sphingolipids that are sphingosine and ceramide derivatives can be generated with hydroxyl-replacement groups that block the bioconversion of ceramide to sphingolipids such as sphingomyelin, ceramide-1-phosphate, sphingosine, sphingosine-1-phosphate and glucosylceramide and thus result in enhanced intracellular ceramide content. Many such derivatives are detailed in Pei, et al., WO 95/21175 and U.S. Pat. No. 5,681,589 are incorporated herein by reference. Studies have indicated that this is possible without inhibiting the signaling properties of the ceramide molecule. By inhibiting the metabolism of ceramide, the metabolic stability of the lipid can be increased thereby stimulating apoptosis. Either hydroxyl group may be modified with a hydroxyl-replacement group. Any hydroxyl-replacement group that can effectively inhibit conversion of ceramide to one or more metabolites can be employed. Replacement of the amide moiety by a sulfonamide-group is one possibility to enhance the metabolic stability of the lipid. Enhanced catabolic stability may likely be achieved by incorporation of this modification into the ceramide structure. This should also prevent formation of sphingosine-1-phosphate which is an additional signaling substance metabolically derived from ceramide.

Types of Delivery Vehicles

Delivery vehicles for use in this invention include lipid carriers, liposomes, lipid inicelles, lipoprotein micelles, lipid-stabilized emulsions, cyclodextrins, polymer nanoparticles, polymer microparticles, block copolymer micelles, polymer-lipid hybrid systems, derivatized single chain polymers, and the like, all containing a sphingolipid and an acid-derivatized sterol. The carriers can be prepared with additional lipid or polymer components conventionally employed in the art. For example, the lipid carriers may comprise surface stabilizing hydrophilic polymer-lipid conjugates such as polyethylene glycol-DSPE, to enhance circulation longevity (see Example 3). Optionally, negatively charged lipids such as phosphatidylglycerol (PG) and phosphatidylinositol (PI) can be included in lipid carrier formulations to increase the circulation longevity of the delivery vehicle. These lipids may be employed to replace hydrophilic polymer-lipid conjugates as surface stabilizing agents. Lipid carriers of the invention may also contain therapeutic lipids in addition to bioactive sphingolipids. Examples include ether lipids, phosphatidic acid, phosphonates and phosphatidylserine.

In addition to the sphingolipid, additional therapeutic agents may be included. Particularly preferred are antitumor agents such as DNA damaging agents, DNA repair inhibitors, topoisomerase I inhibitors, S/G2 and G2/M cell cycle checkpoint inhibitors, G1/early-S checkpoint inhibitors and CDK inhibitors, G2M checkpoint inhibitors, receptor tyrosine kinase inhibitors, apoptosis-inducing agents, cell cycle control inhibitors, hormones and anti-angiogenic agents.

DNA damaging agents include, for example, chlorambucil, carboplatinum and doxorubicin; DNA repair inhibitors include aminopterin derivatives, 5-fluorouracil, and methotrexate; topoisomerase I inhibitors include irinotecan and camptothecin; topoisomerase II inhibitors include deoxydoxorubicin and etoposide; S/G2 and G2/M checkpoint inhibitors include bleomycin and dolastatin; G1/early-S checkpoint and cyclin dependent kinase inhibitors include flavopiridol and hydroxyurea; G2/M checkpoint inhibitors include bleomycin and vincristine; receptor tyrosine kinase inhibitors include AG-1478 and lavendustin A. Various other therapeutic compounds could also be included; however, the combined effect of a sphingolipid and an antitumor agent on tumor cells is particularly advantageous.

Tumors that can be treated include lymphomas, carcinomas, and solid tumors of various organs.

Delivery vehicles of the invention may further comprise targeting ligands. Such ligands may be individually incorporated into the delivery vehicle or conjugated to components comprising the delivery vehicle, such as lipids or polymers. “Targeting ligands” are molecules, typically naturally-occurring, that bind to cell-surface moleoules in both specific and non-specific interactions. Non-specific binding is generally through charge-charge interactions, whereas specific binding occurs through cell-surface receptors. This type of receptor-ligand binding can initiate intemalization-processes. Internalization can occur through phagocytosis, endocytosis or receptor-mediated endocytosis. Preferably, ligands are chosen such that they bind to specific cell-surface receptors known as “internalizing receptors.” Binding to these receptors leads to receptor-mediated endocytosis which results in the receptor, ligand and any ligand-associated material being internalized within an endosome or lysosome of the cell. In this way, targeting ligands attached to delivery vehicles of the invention will thus result in enhanced delivery of the sphingosine-containing delivery vehicles to the low pH environment of the endosome or lysosome. This will lead to destabilization of the delivery vehicle due to protonation of the associated acid-derivatized sterol which will allow for increased intracellular delivery of the sphingosine. The use of targeting ligands aimed at internalizing receptors is particularly useful for cancer treatment as many of these receptors are overexpressed on cancer cells. Several studies of different cancers, including breast cancer, have correlated expression of the transferrin receptor.(TfR) to tumor grade and metastatic potential. Other non-limiting examples of internalizing receptors are low-density lipoprotein receptor (LDL-R), epidermal growth factor receptor (EGF-R), folate receptor (FR), and cluster designation (CD) molecules, such as CD3.

The targeting agents may be ligands specific for cell surface receptors, immunoglobulins or fragments thereof, and the like. These targeting agents can be coupled to the delivery vehicles using methods generally known in the art.

Preferred lipid carriers for use in this invention are liposomes. Liposomes can be prepared as described in Liposomes: Rational Design (A.S. Janoff, ed., Marcel Dekker, Inc., New York, N.Y., or by additional techniques known to those knowledgeable in the art. Suitable liposomes for use in this invention include large unilamellar vesicles (LUVs), multilamellar vesicles (MLVs), small unilamellar vesicles (SUVs) and interdigitating fusion liposomes.

Sphingolipids and acid-derivatized sterols may be prepared as liposomes of the invention using standard methods described in the art. Said liposomes may further comprise one or more lipids commonly used in the preparation of liposomes as well as other non-lipid components. It should be readily apparent to those knowledgeable in the art that a number of lipid combinations could be employed to generate delivery vehicles of the present invention.

The internal compartment of the liposomes can optionally encapsulate one or more therapeutic agents. This provides for the preparation of a delivery vehicle that contains encapsulated therapeutic agents in addition to a bioactive sphingolipid, thereby allowing for the treatment of diseases that result from multiple molecular mechanisms. This is of particular significance as synergistic effects between exogenous ceramide and anticancer treatments have been reported (Mehta, et al., Cancer Chemother. Pharmacol. (2000) 46:85-92).

Various methods may also be utilized to encapsulate active agents in liposomes. Examples of suitable loading techniques include conventional passive and active entrapment methods. Passive methods of encapsulating active agents in liposomes involve encapsulating the agent during the preparation of the liposomes. This includes a passive entrapment method described by Bangham, et al. (J. Mol. Biol. (1965) 12:238). This technique results in the formation of multilamellar vesicles (MLVs) that can be converted to large unilamellar vesicles (LUVs) or small unilamellar vesicles (SUVs) upon extrusion. Additional suitable methods of passive encapsulation include an ether injection technique described by Deamer and Bangham (Biochim. Biophys. Acta (1976) 443:629) and the Reverse Phase Evaporation technique as described by Szoka and Paphadjopoulos (P.N.A.S. (1978) 75:4194).

Active methods of encapsulation include the pH gradient loading technique described in U.S. Pat. Nos. 5,616,341, 5,736,155 and 5,785,987. A preferred method of pH gradient loading is the citrate-base loading method utilizing citrate as the internal buffer at a pH of 4.0 and a neutral exterior buffer. Other methods employed to establish and maintain a pH gradient across a liposome involve the use of an ionophore that can insert into the liposome membrane and transport ions across membranes in exchange for protons (see U.S. Pat. No. 5,837,282). A recent technique utilizing transition metals to drive the uptake of drugs into liposomes via complexation in the absence of an ionophore may also be used. This technique relies on the formation of a drug-metal complex rather than the establishment of a pH gradient to drive uptake of drug.

Passive and active methods of entrapment may also be coupled in order to prepare a liposome formulation containing more than one encapsulated agent. pH sensitive liposomes are dissolved in Drummond, D et al Prog Lipid Res. (2000) 39: 409-460.

Therapeutic Uses of Sphingolipid-Containing Delivery Vehicle Compositions

These delivery vehicle compositions may be used to treat a variety of diseases or conditions in warm-blooded animals and in avian species. Examples of medical uses of the compositions of the present invention include treating cancer, treating cardiovascular diseases such as hypertension, cardiac arrhythmia and restenosis, treating bacterial, viral, fungal or parasitic infections, treating and/or preventing diseases through the use of the compositions of the present inventions as vaccines, treating inflammation or treating autoimmune diseases.

In one embodiment, delivery vehicle compositions in accordance with this invention are preferably used to treat neoplasms. Delivery of formulated sphingolipids to a tumor site is achieved by administration of liposomes or other particulate delivery systems. Preferably liposomes have a diameter of less than 200 nm. Tumor vasculature is generally leakier than normal vasculature due to fenestrations or gaps in the endothelia. This allows the delivery vehicles of 200 nm or less in diameter to penetrate the discontinuous endothelial cell layer and underlying basement membrane surrounding the vessels supplying blood to a tumor. Selective accumulation of the delivery vehicles into tumor sites following extravasation leads to enhanced sphingolipid delivery and therapeutic effectiveness.

Other cosmetic or diagnostic uses, depending upon the particular properties of a preparation, may be envisioned by those skilled in the art. For example, because of their sensitivity to divalent cations, cholesteryl hemisuccinate liposomes of the present invention may be made to entrap indicator dyes which are sensitive to divalent cations for use in colorimetric diagnostic assays.

Administering Delivery Vehicle Compositions

As mentioned above, the delivery vehicle compositions of the present invention may be administered to warm-blooded animals, including humans as well as to domestic avian species. For treatment of human ailments, a qualified physician will determine how the compositions of the present invention should be utilized with respect to dose, schedule and route of administration using established protocols. Such applications may also utilize dose escalation should agents encapsulated in delivery vehicle compositions of the present invention exhibit reduced toxicity to healthy tissues of the subject.

Preferably, the pharmaceutical compositions of the present invention are administered parenterally, i.e., intraarterially, intravenously, intraperitoneally, subcutaneously, or intramuscularly. More preferably, the pharmaceutical compositions are administered intravenously or intraperitoneally by a bolus injection. For example, see Rahman, et al., U.S. Pat. No. 3,993,754; Sears, U.S. Pat. No. 4,145,410; Papahadjopoulos, et al., U.S. Pat. No. 4,235,871; Schneider, U.S. Pat. No. 4,224,179; Lenk, et al., U.S. Pat. No. 4,522,803; and Fountain, et al., U.S. Pat. No. 4,588,578, incorporated by reference.

In other methods, the pharmaceutical or cosmetic preparations of the present invention can be contacted with the target tissue by direct application of the preparation to the tissue. The application may be made by topical, “open” or “closed” procedures. By “topical”, it is meant the direct application of the sphingolipid preparation to a tissue exposed to the environment, such as the skin, oropharynx, external auditory canal, and the like. “Open” procedures are those procedures that include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin. For example, the preparations may be administered to the peritoneum by needle lavage. Alternatively, the preparations may be administered through endoscopic devices.

Pharmaceutical compositions comprising delivery vehicles of the invention are prepared according to standard techniques and may comprise water, buffered water, 0.9% saline, 0.3% glycine, 5% dextrose and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. Additionally, the delivery vehicle suspension may include lipid-protective agents which protect lipids against free-radical and lipid-peroxidative damages on storage. Lipophilic free-radical quenchers, such as alpha-tocopherol and water-soluble iron-specific chelators, such as ferrioxamine, are suitable.

The concentration of delivery vehicles in the pharmaceutical formulations can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment. Alternatively, delivery vehicles composed of irritating lipids may be diluted to low concentrations to lessen inflammation at the site of administration. For diagnosis, the amount of delivery vehicles administered will depend upon the particular label used, the disease state being diagnosed and the judgment of the clinician.

Preferably, the pharmaceutical compositions of the present invention are administered intravenously. Dosage for the delivery vehicle formulations will depend on the ratio of drug to lipid and the administrating physician's opinion based on age, weight, and condition of the patient.

In addition to pharmaceutical compositions, suitable formulations for veterinary use may be prepared and administered in a manner suitable to the subject. Preferred veterinary subjects include mammalian species, for example, non-human primates, dogs, cats, cattle, horses, sheep, and domesticated fowl. Subjects may also include laboratory animals, for example, in particular, rats, rabbits, mice, and guinea pigs.

The following examples are offered to illustrate but not to limit the invention.

### EXAMPLES

Materials

All phospholipids and ceramides were obtained from Avanti Polar Lipids (Alabaster, AL). Cholesterol, cholesteryl hemisuccinate and MTT reagent were obtained from Sigma-Aldrich Canada (Oakville, ON, Canada). [³H]cholesterylhexadecyl ether ([³H]CHE) was purchased from Perkin Elmer (Boston, Mass.). [¹⁴C]C₆- and [¹⁴C]C₁₆-ceramide were purchased from American Radiolabeled Chemicals (St. Louis, Mo.). Dulbecco's Modified Eagle's Medium (DMEM) and Hank's Balanced Salt Solution (without pH indicator; Hank's) were obtained from Stem Cell Technologies (Vancouver, BC, Canada). Fetal bovine serum was purchased from Hyclone (Logan, Utah). L-glutamine and trypsin-EDTA were obtained from Gibco BRL (Burlington, ON, Canada). The Micro BCA Protein Assay kit was purchased from Pierce (Rockford, Ill.). Tissue culture flasks, incubation plates and cell scrapers were obtained from Falcon (Becton Dickinson, Franklin Lakes, N.J.).

Cell Lines and Culture

Human estrogen receptor negative MDA435/LCC6 wild-type and MDR-1 gene transfected MDA435/LCC6^{MDR1} multidrug resistant breast cancer cell lines were obtained from Dr. Robert Clark, Georgetown University, Washington, D.C. J774 murine macrophage cells were obtained from ATCC (Rockville, Md.). All cells were grown as adherent monolayer cultures in 25-cm² Falcon flasks in DMEM supplemented with 10% fetal bovine serum and 1% L-glutamine. Cells were maintained at 37° C. in humidified air with 5% CO₂. Cells were sub-cultured weekly using 0.25% trypsin with 1 mM EDTA (MDA435/LCC6) or gentle cell scraping (J774).

Prearation of Liposomes

Lipids were weighed into individual test tubes and dissolved in 1 mL of chloroform (DPPC, DSPC, CHEMS, Chol), ethanol (C₂-, C₆-, C₈-, C₁₀-, C₁₄-ceramide) or chloroform:methanol (2:1, volume/volume; C₁₆-ceramide). C₁₆-ceramide required brief heating at 65° C. to achieve complete dissolution. Appropriate volumes of each lipid were transferred to a single tube in order to achieve the desired ratio of each lipid component. All ratios indicated are on a mole:mole basis. [³H]CHE was incorporated at 1 μCi/mg lipid as a non-exchangeable, non-metabolizeable lipid marker to facilitate liposome quantitation. For the preparation of ceramide-containing liposomes, [¹⁴C]C₆-ceramide or [¹⁴C]C₁₆-ceramide was incorporated into the formulation at 0.5 μCi/mg ceramide. The mixtures were evaporated with vortexing and heating under a stream of nitrogen gas and subjected to vacuum drying for a minimum of 4 hours to produce a homogenous lipid film. The lipid film was hydrated in 1 ml of warm Hepes buffered saline (HBS; 20 mM Hepes/150 mM NaCl; pH 7.4) with vortexing. Homogenously sized liposomes were then produced following a 10 cycle extrusion through three stacked 100 nm polycarbonate filters (Nucleopore, Canada) at 65° C. for non-ceramide formulations and 95° C. for ceramide formulations, using an extrusion apparatus (Lipex Biomembranes, Vancouver, BC, Canada). The resulting mean liposome diameter obtained following extrusion was within a range of 91-132 nm, depending on lipid composition, as determined by quazi-elastic light scattering using the Nicomp 270 submicron particle sizer model 370/270. Liposome and ceramide concentrations were determined by liquid scintillation counting.

Cytotoxicity Assays

Cell suspensions were diluted 1:1 with trypan blue, counted with a hemocytometer and seeded into 96-well microtitre plates at 1.5×10⁶ cells/well in 0.2 ml complete medium. The perimeter wells were not used and contained 0.2 ml sterile water. The cells were allowed to adhere for 24 hours at 37° C., after which the medium was aspirated and replaced with 0.1 ml fresh medium. Free ceramide, control liposome or ceramide-liposome stocks were diluted into complete medium and added to cells in 0.1 ml to achieve the desired final concentration. The C₁₆-ceramide stock was kept warm and diluted into warm medium prior to addition to the cells, and remained in solution at all times. After 72 hours the cell viability was assessed using a conventional 3-(4,5-dimethylthiazol-2-yl)-2,5-diphenyl tetrazolium bromide (MTT) dye reduction assay. Fifty microliters of 5 mg/ml MTT reagent in phosphate buffered saline (PBS) was added to each well. Viable cells with active mitochondria reduce the MTT to an insoluble purple formazan precipitate that is solubilized by the subsequent addition of 150 μl dimethyl sulfoxide. The formazan dye was measured spectrophotometrically using a Dynex plate reader (570 nm). All assays were performed in triplicate. The cytotoxic effect of each treatment was expressed as percent cell viability relative to untreated control cells (% control) and is defined as: [(Abs₅₇₀ treated cells)/( Abs₅₇₀ control cells)]×100.

Lipid Uptake Studies

Cell suspensions were diluted 1:1 with trypan blue, counted with a hemocytometer and seeded into 6-well Falcon plates at 2.5×10⁵ cells/well in 2 ml complete medium. The cells were allowed to adhere for 24 hours at 37° C., after which the medium was aspirated and replaced with 1 ml complete medium. Free ceramide, control liposome or ceramide-liposome stocks were diluted in 1 ml complete medium and added to each well to give the desired final concentration. Cells were incubated with the treatments for 1, 4 and 24 hours at 37° C. The incubation medium was then aspirated and cells were washed twice with 2 ml Hank's. Cells were gently scraped into 0.5 ml Hank's and collected into glass scintillation vials using glass pipettes. Each well was rinsed with an additional 0.5 ml Hank's to remove residual cells. An aliquot of cells was removed for protein quantification and the remainder was counted for radioactivity by scintillation counting.

Spectrophotometric Protein Quantification

The protein content of each cell aliquot was determined using the Pierce micro BCA protein assay according to the method included with the assay kit, Briefly, a standard curve was prepared using the supplied purified bovine serum albumin (BSA) diluted in distilled water to a final volume of 0.5 ml. Samples were prepared using 5 μl cell suspension+495 μl dH₂O. Micro BCA reagents A, B and C were added in the specified ratios. All samples and standards were prepared in glass test tubes, which were heated in a 65° C. waterbath for 1 hour and cooled to room temperature. The absorbance at 562 nm of each sample was read against a dH₂O reference. The protein concentration for each cell sample was determined using a standard curve prepared from the known BSA samples.

In Vivo Evaluation of Antitumor Activity

Evaluation of the antitumor activity of C₁₆-ceramide-containing liposomes was carried out in the J774 ascites tumor model. On day zero, 1×10⁶ cells were inoculated intraperitoneally (i.p.) into female Balb/c mice (12 mice/group). Mice were administered saline control and liposomes either by intravenous bolus or intraperitoneally on the days indicated in the Examples below. Animals were weighed and monitored daily for survival.

### Example 1

### The Inactivity of Free Long-Chain Ceramides is Due to Lack of Intracellular Delivery

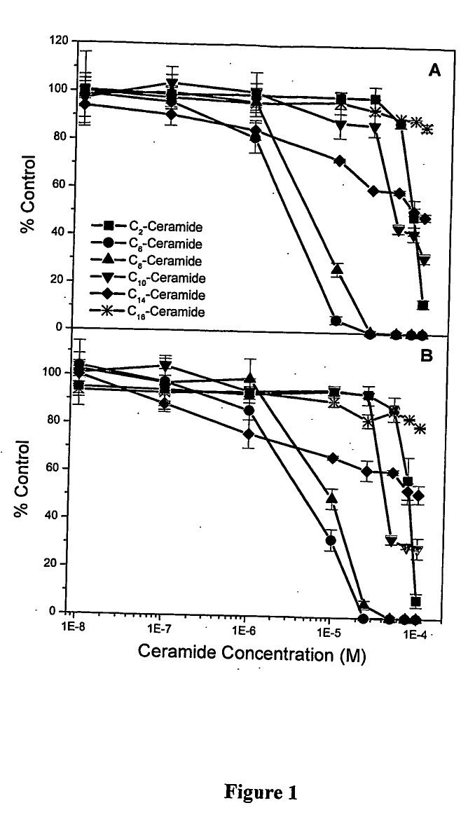

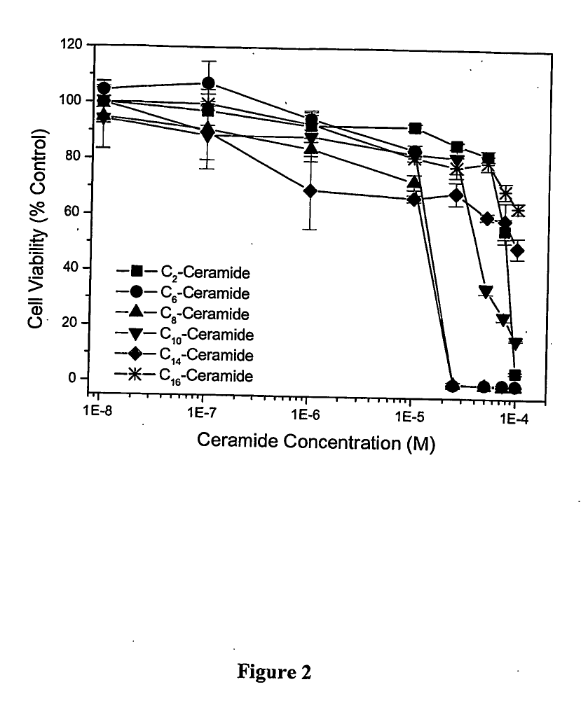

In order evaluate the relationship between the acyl chain length of exogenous ceramides and cytotoxicity, the MTT assay (see Methods) was carried out by the addition of free C₂-, C₆-, C8-, C₁₀- and C₁₆-ceramide to human estrogen-negative MDA435/LCC6 wild-type and MDR-1 gene transfected MDA435/LCC6^{MDR1} breast cancer cells, over a range of 0-100 μM final ceramide concentration. The 72-hour MTT cytotoxicity results shown in FIG. 1 demonstrate that cytotoxic activity is dependent on ceramide acyl chain length. With the exception of C₂-ceramide, as acyl chain length increased the cytotoxic activity decreased. This trend may be explained by the hydrophobicity, and thus cell-permeability characteristics, of the various ceramides. The C₂-ceramide, being very hydrophilic, likely remains dispersed in the tissue culture media. The C₆- and C₈-ceramides were the most cytotoxic, with IC₅₀ values in the 3-14 μM range. As the chain length increases to C₁₀-, C₁₄- and C₁₆-ceramide the hydrophobic nature also increases, and IC₅₀ values of approximately 45 μM are observed for C₁₀-ceramide and in excess of 100 μM are observed for C₁₄- and C₁₆-ceramides. From these results, it was concluded that the C₆-ceramide is the most potent exogenous ceramide form. Free ceramides of varying acyl chain length were also tested for cytotoxicity in the J774 macrophage cell line. Similar results were obtained in the J774 cell line as with the wild-type and resistant MDA435/LCC6 cell line, with increases in acyl chain length correlating with decreases in cytotoxicity (see FIG. 2).

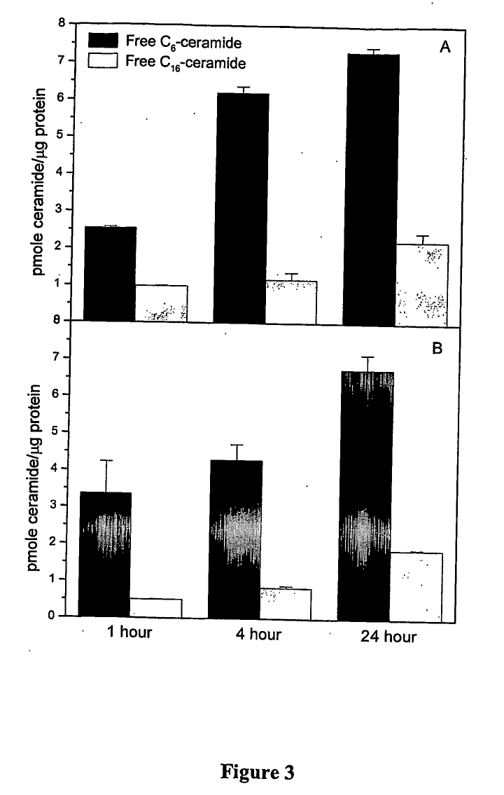

Cellular uptake studies were performed using radioactive C₆- and C₁₆-ceramides to demonstrate that acyl chain length determines ceramide cytotoxicity due to differences in cell-permeability. Uptake studies were performed as set forth in the Methods employing the MDA435/LCC6 and MDA435/LCC6^{MDR1} cell lines. FIG. 3 shows that for both the wild-type and resistant cell lines [¹⁴C]C₁₆-ceramides levels steadily increased to 7 pmole ceramide/μg protein over the 24 hour incubation period while [¹⁴C]C₁₆-ceramide levels did not significantly increase and remained under 2 pmole ceramide/μg protein over the 24 hour period. The lack of cell-associated [¹⁴C]C₁₆-ceramide suggests that the long chain ceramide does not partition into the cell membrane. It is possible that these lipids form aggregate structures or bind to serum proteins in the culture medium, although no turbidity was observed. Nevertheless, the C₁₆-ceramide was not found to be cell-associated, which correlates with its lack of cytotoxic effect.

### Example 2

### The Intracellular Delivery of Long-Chain Ceramides is Enhanced In Vitro by Formulation in Cholesteryl Hemisuccinate (CHEMS) Liposomes

Although C₆-ceramide is efficacious in vitro, C₁₆-ceramide is the more physiologically relevant ceramide. This is based on the observation that both short- and long-term increases in C₁₆-ceramide accumulation have been observed during apoptosis (Thomas, et al. (supra)). As the results in Example 1 suggest that the lack of cytotoxic effect of C₁₆-ceramide may be due to poor cellular uptake, C₁₆-ceramide was formulated into liposomes with the goal of increasing the intracellular delivery of this lipid.

Liposomes containing various lipid components were made as described in the Methods section and the formulation characteristics were monitored during preparation. The results are presented in Table 1 below:

TABLE 1

| | Mole % | |
|---|---|---|
| Lipid Composition | C₁₆-Cer | Formulation Characteristics |
| C₁₆-cer/DSPC/Chol (15:40:45) | 15 | hydrates well and extrudes with >80% |
| | | efficiency |
| C₁₆-cer/DSPC/Chol (20:35:45) | 20 | lipid film difficult to hydrate; lipid |
| | | aggregates |
| C₁₆-cer/DSPC/Chol (15:55:30) | 15 | hydrates well and extrudes with >80% |
| | | efficiency |
| C₁₆-cer/DSPC/Chol (20:50:30) | 20 | lipid film difficult to hydrate; lipid |
| | | aggregates |
| C₁₆-cer/Chol (50:50) | 50 | Lipid film difficult to hydrate; lipid |
| | | aggregates |
| C₁₆-cer/CHEMS (50:50) | 50 | Hydrates well and extrudes with >80% |
| | | efficiency at concentration ≦10 mg/mL |
| C₁₆-cer/DPPG/PEG350-DSPE | 30 | Hydrates well and extrudes but non- |
| (30:30:40) | | uniform trimodal size distribution; liposome |
| | | aggregate |
| C₁₆-cer/DSPC/DOPE/PEG2000- | 20 | Lipid film difficult to hydrate; lipid |
| DSPE (20:35:35:10) | | aggregates |
| C₁₆-cer/DPPC/PEG2000-DSPE | 15 | Hydrates well and extrudes with >80% |
| (15:75:10) | | efficiency |

As depicted in Table 1, liposomes consisting of C₁₆-cer/DSPC/Chol (15:40:45 and 15:55:30 mole ratio) could be prepared with 15 mole % C₁₆-cer. When the mole % of C₁₆-ceramide in these formulations was increased to 20 mole %, the lipid films were difficult to hydrate and lipid aggregates formed. Similarly, liposome formulations containing C₁₆-cer/Chol (50:50 mole ratio), C₁₆-cer/DPPG/PEG350-DSPE (30:30:40 mole ratio) and C₁₆-cer/DSPC/DOPE/PEG2000-DSPE (20:35:35:10 mole ratio) could not be successfully formulated. In contrast, C₁₆-ceramide could be incorporated into CHEMS containing liposomes up to 50 mole %, for a final liposome composition of C₁₆-ceramide/CHEMS of 50:50 (mole ratio). These liposomes were stable and displayed a mean diameter range of 97-132 nm.

The inventors next determined whether the stable formulation of C₁₆-ceramide into CHEMS liposomes translated into enhanced cytotoxicity in vitro. Liposomes consisting of C₁₆-cer/CHEMS (50:50) and control liposomes consisting of DPPC/CHEMS (50:50) were tested for cytotoxic effects in the J774 macrophage cell line by employing the MTT cytotoxicity assay as described in the Methods. As well as providing for the stable formulation of C₁₆-cer, the presence of the pH sensitive CHEMS lipid, imparts to the liposomes the ability to be destabilized in the acidic environment of endosomes and lysosomes. This is due to the protonation of the lipid when exposed to low pH conditions. Thus, J774 macrophage cells were employed due to the ability of this cell line to endocytose liposomes thereby ensuring that the ceramide would be specifically delivered to the endosome. Delivery of ceramide to the endosome is preferred as it is within this membrane compartment that natural ceramide is produced during the process of apoptosis.

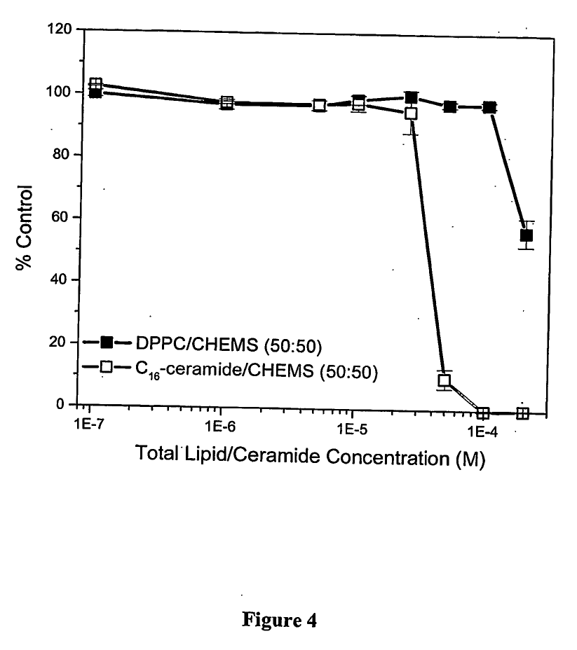

The MTT cytotoxicity results shown in FIG. 4 indicate that liposomes composed of C₁₆-cer/CHEMS (50:50) dramatically improve the cytotoxicity of C₁₆-ceramide in the J774 macrophage cell line. Specifically, while the IC₅₀ value of C₁₆-ceramide when exogenously applied to cells in its free form was well in excess of 100 μM in these cells, its formulation and delivery via CHEMS liposomes decreased its IC₅₀ to 36.1 μM, bringing it into the range of cytotoxicity observed with free C₆-ceramide (14.4 μM). This cytotoxic effect was ceramide-specific and was not attributed to the CHEMS lipid alone, as control DPPC/CHEMS (50:50) liposomes were non-cytotoxic. The non-ceramide lipid of the control liposomes in this case was DPPC in order to closely match the 16 carbon acyl chain length of the ceramide

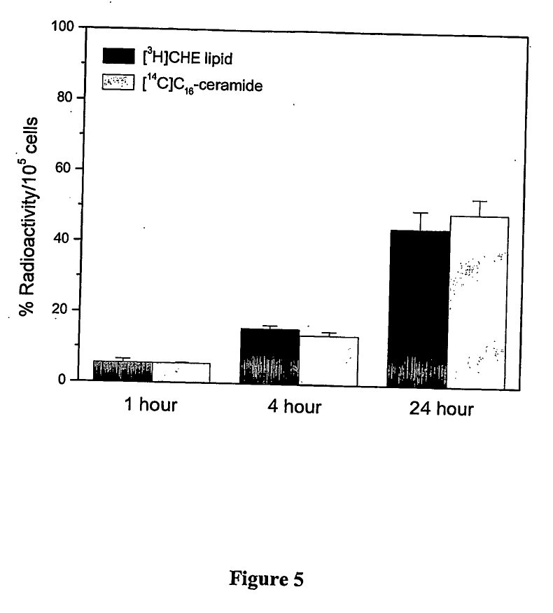

Cellular uptake studies were conducted as described in the Methods. The results indicate that both the liposome and the ceramide components are internalized, as evidenced by uptake of the [³H]CHE and [¹⁴C]C₁₆-ceramide labels which both approach 50% after 24 hours (FIG. 5). This indicates that the C₁₆-ceramide lipid is being delivered via liposomes (rather than by passive lipid exchange), and demonstrates that endosomal delivery of these liposomes can enhance ceramide-induced apoptosis. Thus, in addition to providing a formulation that can stably incorporate up to 50 mole % of the long-chain C₁₆-ceramide, CHEMS also allows for increased cytotoxicity in J774 macrophage cell lines due to increased cellular uptake.

### Example 3

### The In Vivo Anti-Tumor Activity of Long-Chain Ceramides is Enhanced by Formulation in CHEMS Liposomes

In order to determine whether the enhanced in vitro cytotoxicity of C₁₆-ceramide in CHEMS-containing liposomes would also be observed in vivo, the antitumor activity of C₁₆-cer/CHEMS/PEG2000-DSPE (47.5:47.5:5) liposomes containing both [³H]CHE and [¹⁴C]C₁₆-ceramide radiolabels was evaluated in the J774 ascites tumor model and compared to control CHEMS-containing liposomes prepared in the absence of C₁₆-ceramide (DPPC/CHEMS/PEG2000-DSPE, 47.5:47.5:5) and containing the [³H]CHE radiolabel. The lipid, DSPE-PEG2000 was incorporated into the liposomes in order to enhance the blood stability of the formulations.

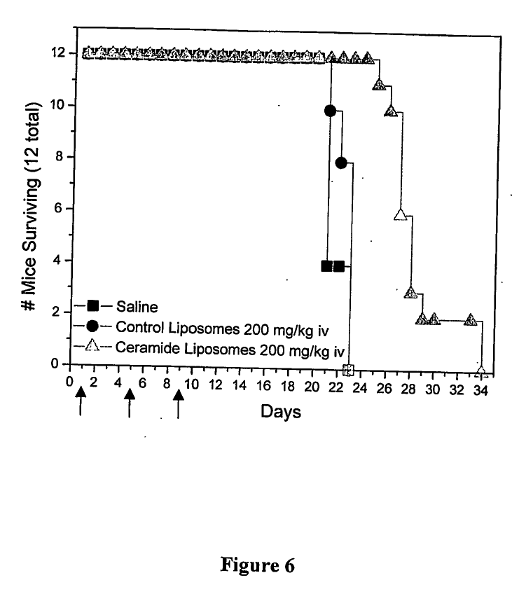

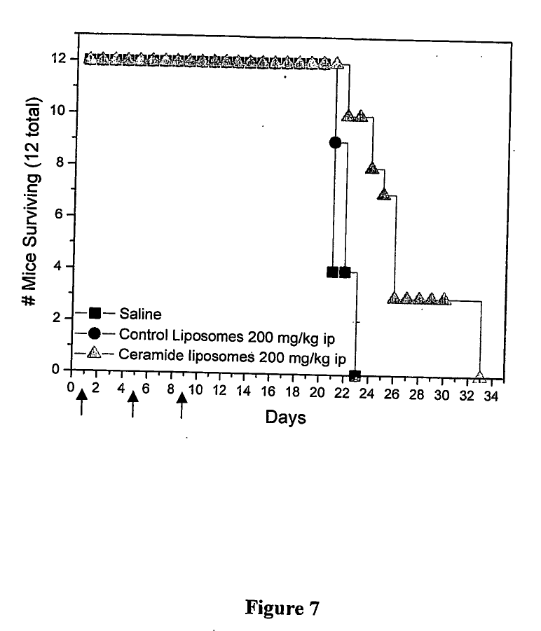

Female Balb/c mice bearing the J774 ascites tumor were prepared as described in the Methods. Two studies were conducted where twelve mice were administered saline, C₁₆-cer/CHEMS/PEG2000-DSPE liposomes and DPPC/CHEMS/PEG2000-DSPE (control) liposomes as follows: a) intravenous bolus on days 1, 5 and 9 at lipid concentrations of 200 mg/kg; and b) intraperitoneally on days 1, 5 and 9 at a lipid concentration of 200 mg/kg for both ceramide-containing liposomes and control liposomes. Results from these studies are presented in FIGS. 6 and 7, respectively, and the arrows in the figures indicate the days of treatment administration.

As demonstrated in FIG. 6, the C₁₆-ceramide-containing formulations displayed increased antitumor effects in the J774 ascites tumor model in relation to control liposomes and saline when administered at a dose of 200 mg/kg by i.v. bolus on days 1, 5 and 9 (cell inoculation day 0). Under these treatment conditions, the saline and liposome control groups displayed median survival times of 21.7 and 22.5 days, respectively, while the C₁₆-ceramide containing liposome treatment group had a median survival time of 28.3 days. This corresponded to a statistically significant (p<0.001) mean increase in lifespan (ILS) of 25.6% (FIG. 6).

Direct intraperitoneal administration of the formulations to the site of the ascites tumor cells was carried out in order to investigate whether the therapeutic response might be improved beyond that observed following i.v. administration. In this study, saline, control liposomes and ceramide-containing liposomes were administered i.p. at a 200 mg/kg total lipid dose on days 1, 5, and 9. The survival curves presented in FIG. 7 indicate that groups treated with saline or control liposomes had a median survival time of 21.6 and 22.1 days, respectively. The group treated i.p. with ceramide-liposomes at a dose of 200 mg/kg total lipid showed a median survival time of 26.7 days, which corresponded to a statistically significant (p<0.001) mean ILS of 26.7%.

Cumulatively, these results thus demonstrate that increases in the survival rate of mice bearing the J774 ascites tumor can be obtained by the administration of liposomal formulations comprising C₁₆-ceramide and CHEMS.

1. A composition which comprises delivery vehicles, said delivery vehicles having stably associated therewith at least one acid-derivatized sterol and at least one sphingolipid.

2. The composition of claim 1 wherein said acid-derivatized sterol is an organic acid derivative of a sterol.

3. The composition of claim 2 wherein said sterol is cholesterol.

4. The composition of claim 3 wherein said acid-derivatized sterol is cholesteryl hemisuccinate.

5. The composition of claim 1 wherein said sphingolipid is hydrophobic.

6. The composition of claim 1 wherein said sphingolipid contains at least 6 carbon atoms in at least one acyl chain.

7. The composition of claim 1 wherein said sphingolipid is a sphingosine or a derivative thereof.

8. The composition of claim 7 wherein said sphingolipid is a sphingosine derivative that inhibits ceramide metabolism.

9. The composition of claim 7 wherein said sphingolipid is a ceramide or a derivative thereof.

10. The composition of claim 9 wherein said sphingolipid is a ceramide derivative that inhibits ceramide metabolism.

11. A method to deliver an effective amount of at least one sphingolipid into cells which method comprises contacting said cells with the composition of claim 1.

12. A method to prepare a composition which comprises at least one hydrophobic sphingolipid, which method comprises incorporating said sphingolipid into delivery vehicles wherein said delivery vehicles have stably associated therewith at least one acid-derivatized sterol.

13. The composition of claim 1 which further comprises at least one anti-cancer agent.

14. A method to treat cancer in a subject, which method comprises administering to a subject in need of such treatment an effective amount of the composition of claim 1.

15. A method to treat cancer in a subject, which method comprises administering to a subject in need of such treatment an effective amount of the composition of claim 13.
